(19) United States
(12) Patent Application Publication
(10) Pub. No.: US 2019/0033286 A1
(43) Pub. Date: Jan. 31, 2019

(54) REDUNDANT POLYMER ANALYSIS BY TRANSLOCATION REVERSALS

(71) Applicant: Quantapore, Inc., Menlo Park, CA (US)

(72) Inventor: Stuart DAVIDSON, Menlo Park, CA (US)

(73) Assignee: Quantapore, Inc., Menlo Park, CA (US)

(21) Appl. No.: 16/072,466

(22) PCT Filed: Feb. 24, 2017
(86) PCT No.: PCT/US2017/019491

§ 371 Date: Jul. 24, 2018

Related U.S. Application Data
(60) Provisional application No. 62/299,902, filed on Feb. 25, 2016.

(51) Int. Cl. G01N 33/487; B01L 3/00; G01N 21/64

(52) U.S. Cl. CPC G01N 33/48721; B01L 3/502715; B82Y 30/00; G01N 21/6452; G01N 21/6428

(57) ABSTRACT

The invention is directed to methods for carrying out redundant measurements on polymers by reversing translocation of the polymers through nanopores that each have a detection region, thereby permitting signals generated from the same polymer structure at different times to be collected. Such repeated measurements are combined in order to reduce noise in a final determination of the polymer structure. In some embodiments, polynucleotides whose different nucleotides have distinguishable fluorescent labels attached are repeatedly translocated through nanopores of a nanopore array to compile repeated measurements of optical signals from the same segments, which may be combined to make a determination of a nucleotide sequence.

### CROSS-REFERENCE TO RELATED APPLICATIONS

This application claims benefit of priority to U.S. Provisional Patent Application No. 62/299,902, filed on Feb. 25, 2016, the content of which is incorporated herein by reference in its entirety.

### BACKGROUND

DNA sequencing technologies developed over the last decade have revolutionized the biological sciences, e.g. van Dijk et al, Trends in Genetics, 30(9): 418-426 (2014). However, there remains a host of challenges that must be overcome to achieve the full potential of the technology, including reduction of per-run sequencing cost, simplification of sample preparation, reduction of run times, increasing sequence read lengths, improving data analysis, and the like. Single molecule sequencing techniques, such as nanopore-based sequencing, may address some of these challenges; however, these approaches have their own set of technical difficulties, such as, reliable nanostructure fabrication, control of DNA translocation rates, measurements with low signal-to-noise ratios, unambiguous nucleotide discrimination, detection and processing of signals from large arrays of nanoscale sensors, and so on, e.g. Branton et al, Nature Biotechnology, 26(10): 1146-1153 (2008).

In view of the above, it would be advantageous to nanopore sensor technology in general and its particular applications, such as optically based nanopore sequencing, if methods and devices were available that addressed the problem of detecting and measuring weak signals in the presence of large amounts of noise.

### SUMMARY OF THE INVENTION

The present invention is directed to methods and devices for addressing the problems of low signal-to-noise measurements in single molecule analysis using nanopores. In one aspect, methods and devices of the invention are directed to reducing noise by repeated translocation of polymer analytes through nanopores.

In some aspects, the invention is directed to a method of analyzing characteristics of polymers by the following steps: (a) providing a nanopore array wherein each nanopore is capable of providing fluid communication between a first chamber and a second chamber and providing signals related to at least one property of a polymer translocating therethrough and wherein a fraction of nanopores in the nanopore array contains polymers; (b) translocating polymers through nanopores of the nanopore array from the first chamber to the second chamber; (c) detecting forward signals from the translocating polymers; (d) reversing the translocation of polymers; (e) detecting reverse signals from polymers whose translocation through a nanopore was reversed; (f) determining at least one property of each such polymer from the forward and reverse signals.

In other embodiments, the invention is directed to methods of analyzing polynucleotides by the following steps: (a) providing a nanopore array wherein each nanopore is capable of providing fluid communication between a first chamber and a second chamber and providing polynucleotides whose different kinds of nucleotides have different fluorescent labels attached which generate distinguishable optical signals, so that different kinds of nucleotide may be identified by an optical signal from its attached fluorescent label and wherein a fraction of nanopores in the nanopore array are occupied by polynucleotides; (b) translocating polynucleotides through nanopores of the nanopore array in a direction from the first chamber to the second chamber; (c) detecting forward optical signals from the translocating polynucleotides; (d) reversing the direction of translocation of the polynucleotides; (e) detecting reverse optical signals from the polynucleotides whose translocation through a nanopore was reversed; and (f) determining a nucleotide sequence of each polynucleotide from the forward and reverse optical signals.

The present invention advantageously overcomes the problem of low signal-to-noise measurements of characteristics of single polymers in nanopore-based detection systems, particularly those using optically labeled polymers. These and other advantages of the present invention are exemplified in a number of implementations and applications, some of which are summarized below and throughout the specification.

### BRIEF DESCRIPTION OF THE DRAWINGS

FIGS. 1A-1F illustrates elements of the invention in particular embodiments.

FIG. 2 illustrates acquisition and use of redundant data in accordance with one embodiment of the invention.

FIG. 3 illustrates an epi-illumination system that may be used with some embodiments of the invention.

FIG. 4 illustrates an embodiment wherein polymer analytes comprise polymers forming random coils at their ends.

### DETAILED DESCRIPTION OF THE INVENTION

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention. For example, particular nanopore types and numbers, particular labels, FRET pairs, detection schemes, fabrication approaches of the invention are shown for purposes of illustration. It should be appreciated, however, that the disclosure is not intended to be limiting in this respect, as other types of nanopores, arrays of nanopores, and other fabrication technologies may be utilized to implement various aspects of the systems discussed herein. Guidance for aspects of the invention is found in many available references and treatises well known to those with ordinary skill in the art, including, for example, Cao, Nanostructures & Nanomaterials (Imperial College Press, 2004); Levinson, Principles of Lithography, Second Edition (SPIE Press, 2005); Doering and Nishi, Editors, Handbook of Semiconductor Manufacturing Technology, Second Edition (CRC Press, 2007); Sawyer et al, Electrochemistry for Chemists, 2^{nd} edition (Wiley Interscience, 1995); Bard and Faulkner, Electrochemical Methods: Fundamentals and Applications, 2^{nd} edition (Wiley, 2000); Lakowicz, Principles of Fluorescence Spectroscopy, 3^{rd} edition (Springer, 2006); Hermanson, Bioconjugate Techniques, Second Edition (Academic Press, 2008); and the like, which relevant parts are hereby incorporated by reference.

The invention is directed to methods and devices for carrying out redundant analyses of polymers by reversing translocation of the polymers through nanopores that each have a detection region, thereby permitting signals generated from the same polymer structure or segment at different times to be collected. Such repeated signals may then be compared or otherwise processed in order to reduce noise in the signals. In one aspect, the invention employs an array of nanopores each having a detection region where an optical signal is generated whenever a polymer passes therethrough. In some embodiments of this aspect, different monomers are labeled with different optical labels that produce distinguishable optical signals. In some embodiments, polymers are nucleic acid polymers and different kinds of nucleotides are labeled with different optical labels that produce distinguishable optical signals that permit nucleotides to be identified from the optical signals emitted by their optical labels. In some embodiments, polymer analytes are charged and translocation direction is controlled by the direction of an electrical field across the array of nanopores.

In another aspect, polymer analytes of the invention are free of stopping or blocking moieties at their ends to prevent the polymer analyte from exiting one, or either, orifice of a nanopore after insertion. That is, polymer analytes of the invention, in particular, nucleic acid polymer analytes have free ends that may pass freely through a nanopore. In some embodiments, labeled polymer analytes may pass through, or translocate through, a nanopore at a reduced speed (as compared to an unlabeled polymer analyte) due to interactions between the labels and nanopore bore, and/or due to steric constraints caused by the labels or other adducts attached for the specific purpose of reducing translocation speed. Such adducts may be organic molecules having molecular weights in the same range as those of conventional organic dyes, e.g. molecular weights in the range of 200 to 2000 Da, or in the range of 200 to 1200 Da. In these embodiments, sample preparation is greatly simplified by not requiring blocking groups at the ends of polymer analytes, thereby increasing the efficiency and lowering the cost of analysis. As discussed below, during each reversal of translocation direction, some analyte may be lost from an array, but such losses may be mitigated by selecting longer polymer analytes for analysis and by using larger arrays, that is, arrays with larger numbers of nanopores. In some embodiments, as illustrated in FIG. 4, long polymer analytes (400), such as single stranded nucleic acids, form random coils (402) when free in solution, which may or may not include intra-polymer interactions, such as base pairing. In some embodiments, translocation rates of sufficiently long polymer analytes may be reduced by formation of stable random coils by terminal portions of such polymers. While not intending to be bound by theory, it is believed that the greater entropy of a random coil state of a polynucleotide provides a restoring force on an extended state of the polynucleotide as present during translocation, thereby slowing translocation speed. Such random coils may also prevent loss of polymers during translocation reversals, particularly traversing central portions of the polymers. Some embodiments may provide populations of polymer analytes comprising members with lengths of at least 1000 monomers, or with lengths of at least 10,000 monomers. Some embodiments may provide populations of nucleic acid polymers comprising members with lengths of at least 1000 nucleotides, or with lengths of at least 10,000 nucleotides, or with lengths of at least 20,000 nucleotides.

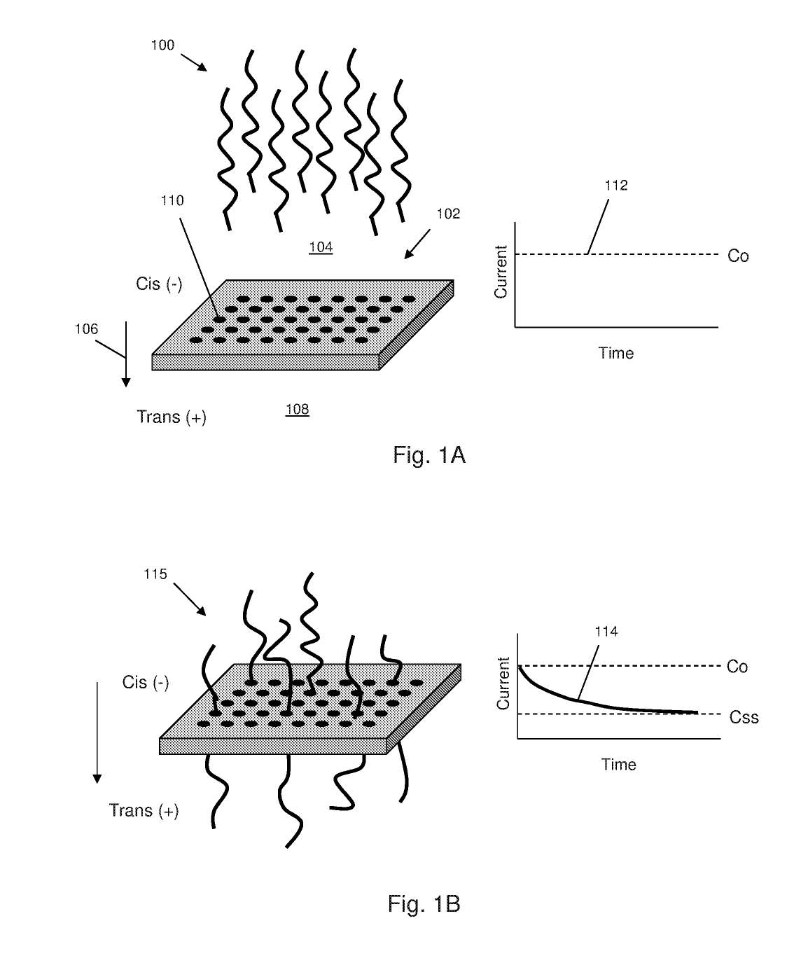

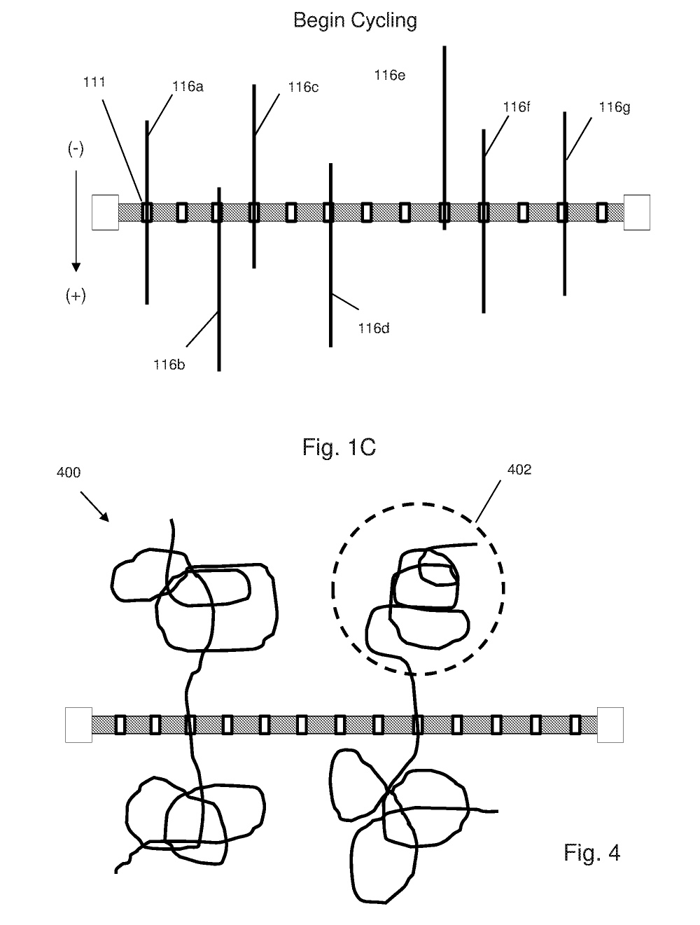

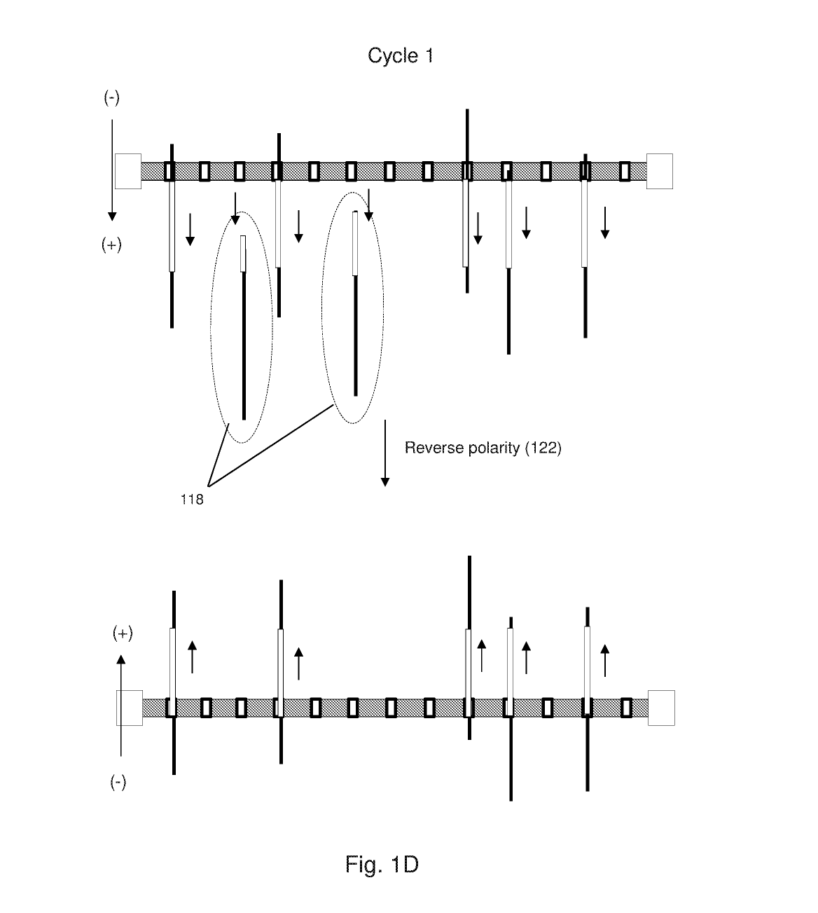

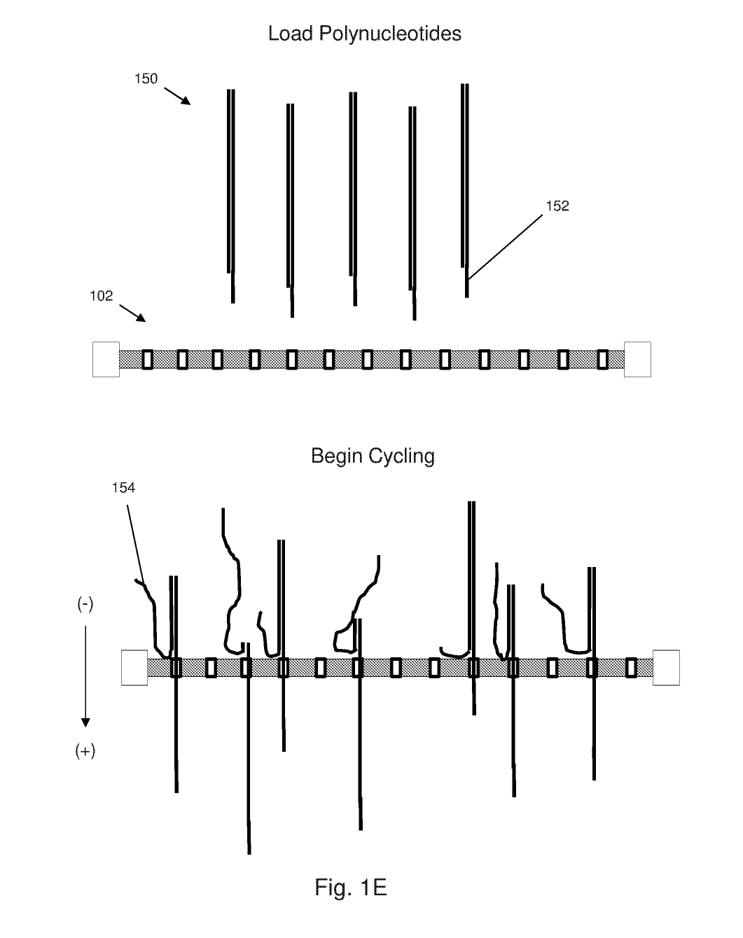

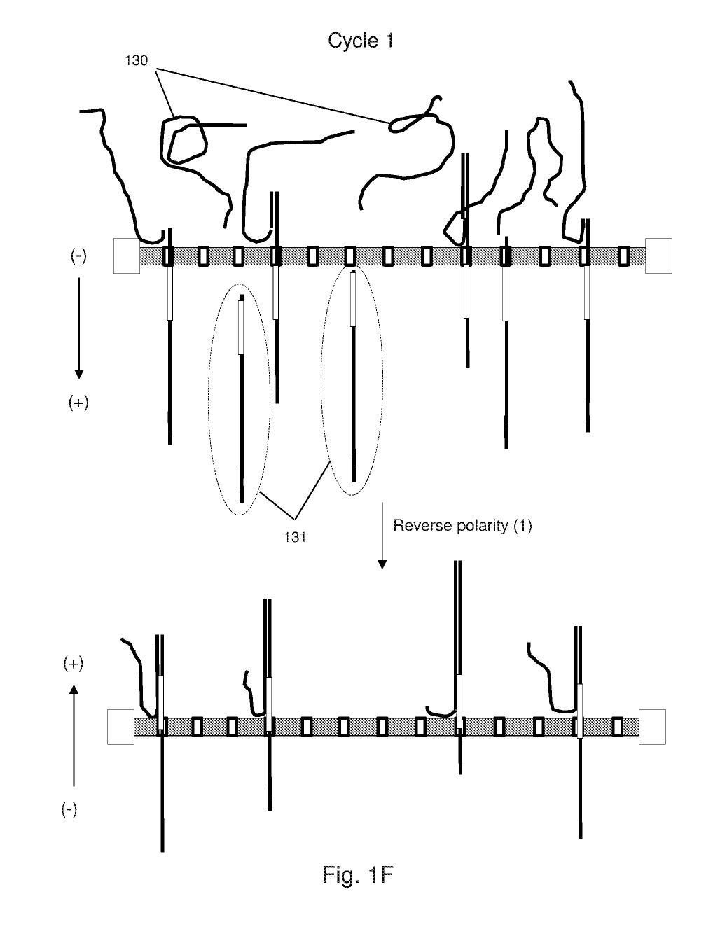

FIGS. 1A-1F illustrate aspects of several embodiments of the invention. In FIG. 1A, negatively charged polymer analytes (100) (for example, single stranded polynucleotides) are exposed to nanopore array (102) in a first chamber (104) under electric field or voltage difference (106) across array (102) that biases the diffusion of polymer analytes (100) to and through nanopores (for example, 110) and into a second chamber (108). Not shown are detection regions associated with each nanopore of array (102) or detector(s) for collecting signal from the detection regions. Detection regions may be selected for generating electrical and/or optical signals. In some embodiments, optical signals are generated in detection regions; for example, FRET signals may be generated by acceptor-labeled polymers passing by a donor-labeled nanopore in a detection region, e.g. such as disclosed in U.S. Pat. No. 8,771,491; International patent publication WO2014/190322; or the like, which are incorporated herein by reference. Alternatively, optical labels may be detected directly from fluorescent labels on the polymer analytes, for example, as disclosed in Huber, U.S. patent publication 2016/012281:2, which is incorporated herein by reference. In this latter case, detection regions are defined by the time interval in which an optical label transitions from a constrained state within a nanopore to a quenched state after exiting the nanopore. Quenching may be accomplished by employing mutually quenching fluorescent labels or extraneous quenching agents, such as random sequence oligonucleotides (e.g. 5-8-mers) with quenching moieties attached.

Quenching agents may comprise any compound (or set of compounds) that under nanopore sequencing conditions is (i) substantially non-fluorescent, (ii) binds to single stranded nucleic acids, particularly single stranded DNA, and (iii) absorbs excitation energy from other molecules non-radiatively and releases it non-radiatively. In some embodiments, quenching agents further bind non-covalently to single stranded DNA. A large variety of quenching compounds are available for use with the invention including, but not limited to, non-fluorescent derivatives of common synthetic dyes such as cyanine and xanthene dyes. Guidance in selecting quenching compounds may be found in U.S. Pat. Nos. 6,323,337; 6,750,024 and like references, which are incorporated herein by reference.

In some embodiments, optionally, the portion of nanopores occupied by polymer analytes (100) may be monitored by measuring the total current through nanopores of array (102). Prior to any insertions, as steady initial current may be recorded (112), then after disposing polymer analytes (at some predetermined concentration) in first chamber (104) under the influence of electric field (106), some average proportion of nanopores in array (102) will be occupied by polymer analyte to produce a drop in the current across array (102) to some steady state value (114). Similarly, in some embodiments, the portion of nanopores occupied by optically labeled polymer analytes may be monitored as a function of total optical signal; that is, an integration or sum of the optical signals collected from all nanopores in an array.

In some embodiments, at such time the polarity of electrical field (106) may be reversed to change the direction the polymer analytes are translocating. In some embodiments, only a single reversal may be made. In other embodiments, multiple reversals may be made for predetermined time intervals. In some embodiments, a plurality of reversals are made after uniform, i.e. equal, time intervals. In such embodiments, for some polymer analytes, the same segment of the polymers will pass through a detection region multiple times. In some embodiments, the plurality of reversal will be an even number. In some embodiments where an even number of reversals are completed the net movement of polymer analytes will be from the first chamber to the second chamber.

FIG. 1C illustrates a cut-away view of nanopore array (102) showing nanopores (111) and polymer analytes (116a-g) (all shown with the same length for convenience) at varying degrees of translocation through their respective nanopores. It is assumed for illustration that at any given time the position of a nanopore along a polymer in a population undergoing translocation is random and equally likely to be anywhere along the polymer's length. Under such conditions, if signal detection begins and proceeds for a time prior to a first reversal of translocation direction, there will be a fraction of polymers that may be lost (for example, polymers 118, in FIG. 1D) and not available for signal detection after the first reversal of translocation direction. Some loss may occur during each cycle of reversals. Segments (120) of the respective polymers from which signals are collected from a detection region immediately prior to reversal (122). If the translocation after reversal moves the polymers through the detection regions to generate and collect signals over the same segments (120), then two sets of signals are collected for characteristics of interest in the segments. For example, if polymers are nucleic acid polymers with acceptor-labeled nucleotides and the trans side (e.g. see FIG. 1A-B) of nanopores comprise associated donor labels, then two sets of data from FRET signals generated between donors and acceptors may be collected. Such data may then be reordered and aligned to increase signal-to-noise ratios of nucleotide and/or sequence calls.

The pattern of translocation reversals, with the objective of obtaining redundant data from the same polymer feature or segment (such as, monomer sequence information), may vary widely; that is, for example, the number of translocation reversals, the time intervals between reversals, and the translocation speeds during time intervals may vary widely. In some embodiments, signal generation and data collection is continuous, so that data for a given polymer is collected from the time it enters a nanopore until the time it completely exits the nanopore. In some embodiments, between an entry time and an exit time of a polymer, at least one translocation reversal is implemented. In other embodiments, between an entry time and an exit time, a plurality of translocation reversals are implemented. In some embodiments, the plurality of translocation reversals is an even number greater than one. In some embodiments, the duration of translocation after a reversal may be the same for all reversals so that redundant data is collected from substantially the same polymer segment. In some embodiments, durations of translocations after reversals may be different. In some embodiments, the different durations of translocations after reversals are predetermined. In some embodiments, reversals of translocation direction are cyclic wherein the reversals and their associated translocation durations are implemented as identical pairs; that is, reversals in translocation direction are implemented in one or more cycles of (i) reversal of translocation direction followed by translocation for a first duration, or time t₁, and (ii) reversal of translocation direction followed by translocation for a second duration, or time t₂. In some embodiments, the first time and the second time are equal. In other embodiments, t₁<t₂, so that polymers progress through nanopores in a rachet-like manner. Selection of first and second translocation times may be based on the translocation method (for example, for negatively charged polymers, such as nucleic acid polymers, strength of electric field), average and standard deviation of polymer lengths, type of signal generated by detection region (for example, FRET signals), whether a signal is generation by a single monomer or a plurality of monomers, and the like. In some embodiments, voltage across a nanopore array may be different for different time intervals between translocation reversals, or it may be varied during such interval, for example, for the purpose of optimizing the occupancy of polymers in the array. Thus, in some embodiments including a step of repeatedly reversing translocation direction may include a pattern of translocation reversals with (i) cyclical changes in translocation durations between reversals and in voltage levels across a nanopore array; (ii) a predetermined series of translocation durations between reversals and voltage levels across a nanopore array, that may be cyclical or non-cyclical; or (iii) translocation durations between reversals and voltage levels that are selected automatically in real-time, for example, to optimize an operational parameter, such as, polymer occupancy of nanopores in the array. Such real-time selection may be in response to a particular size distribution of polymers in a population being analyzed.

FIGS. 1E-1F illustrate an implementation of the invention where nucleic acid polymer analytes are disposed in a first chamber (104) as double stranded DNAs each with a single stranded tail that may be captured by nanopores. In such implementations, nanopores are selected that permit translocations of single stranded DNA but not double stranded DNA; thus, after capture, the double stranded portion is unzipped during translocation. As in the embodiment illustrated above, in this implementation depending on the position of a polymer in a nanopore when reversals are initiated, a polymer may be lost from the nanopore array and made unavailable for further measurements, for example, as illustrated by polymers (130 and 131) in FIG. 1F.

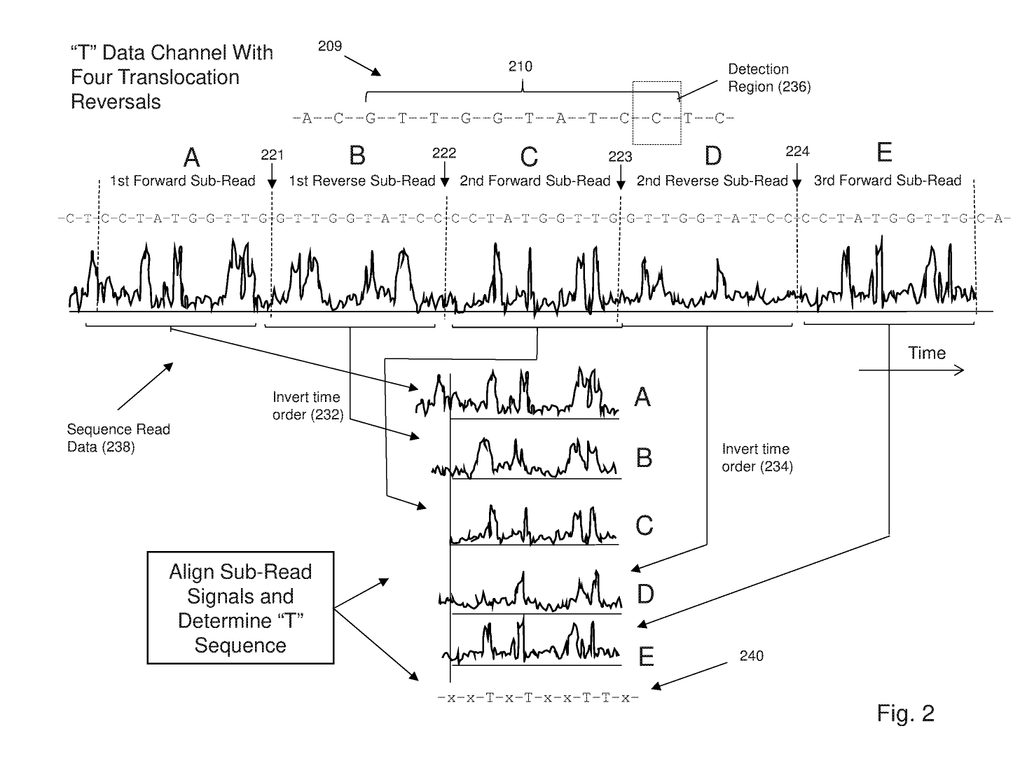

FIG. 2 illustrates how redundant sequence data obtained by methods of the invention may be used to improve sequence analysis of a nucleic acid polymer. In this illustration, segment (210) of acceptor-labeled or fluorescently labeled nucleic acid polymer (209) is passed through a nanopore having detection region (236). In some embodiments, such detection region may comprise a donor that may be excited so that fluorescence resonant energy transfer (FRET) occurs between acceptors on the polynucleotide within a FRET distance of the donor, after which the acceptor emits a fluorescent signal indicative of the nucleotide to which the acceptor is attached. In other embodiments, such detection region may comprise a volume (for example, at the exit of a nanopore) within which fluorescent labels on the polynucleotide may be excited (for example, because of a temporary absence of quenching). In some embodiments, a different and distinguishable acceptor signal or fluorescent signal is generated for each different nucleotide. In FIG. 2, data from signals generated only from “T” nucleotides are shown. The direction of translocation of polymer (209) is reversed at four times marked at (221, 222, 223 and 224) in the illustrated time record of raw data, indicated as “sequence read data” (238) in the figure (which, again, is from only T's). That is, data is shown for three forward signals and two reverse signals. Immediately above the illustrated raw data are copies of segment (210) shown alternately with its sequence in reverse order and in forward (or correct) order (shown as A (1^{st} forward sub-read), B (1^{st} reverse sub-read), C (2^{nd} forward sub-read), D (2^{nd} reverse sub-read), and E (3^{rd} forward sub-read)) to give a “full” sequence read of segment (210) nucleotides as they pass repeatedly through detection region (236). The copies A, B, C, D and E of segment (210) correspond to the illustrated raw data (238). In this illustration, signals from each nucleotide of segment (210) are collected in five separate measurements. Base calls of segment (210) may be obtained from sequence read data (238) by aligning a plurality of sub-read data A, B, C, D or E. In some embodiments, data from only a subset of sub-reads may be combined, for example, only the forward sub-reads (A, C, and E). In other embodiments, both forward and reverse sub-reads may be used by reversing the time ordering of the sub-read data of either the forward or reverse sub-reads (e.g. 232 and 234) prior to aligning so that the underlying nucleotide sequences represented in the data are in the same order. Conventional alignment and data analysis techniques may then be used to generate base calls (240) for segment (210) from the sub-read data. Similar data may be collected and combined for each of distinct signals generated from labeled A's, labeled C's, and labeled G's. In some embodiments, a sequence of a polymer analyte is determined by combining these analyses.

In some embodiments, the invention is directed to a method of analyzing characteristics of polymers by the following steps: (a) providing a nanopore array wherein each nanopore is capable of providing fluid communication between a first chamber and a second chamber and providing signals related to at least one property of a polymer translocating therethrough and wherein a fraction of nanopores in the nanopore array contains polymers that extend from the first chamber to the second chamber; (b) translocating polymers through nanopores of the nanopore array from the first chamber to the second chamber and detecting a forward signal from each translocating polymer; (c) reversing the translocation of polymers and detecting a reverse signal from each polymer whose translocation through a nanopore was reversed; and (d) determining at least one property of each such polymer from the forward and reverse signals. In some embodiments, the step of reversing the translocation direction across a nanopore array may be carried out repeatedly. In such embodiments, repeated reversals of polymer translocation may be continued until either said fraction of nanopores having polymers drops below a predetermined level or said reversals are repeated a predetermined number of times, whichever occurs first. In some embodiments, such fraction may be 5 percent or fewer of nanopores being occupied by polymers and capable of generating signals, or may be 10 percent or fewer of nanopores being occupied by polymers and capable of generating signals. In some embodiments, a predetermined number of reversals may be a plurality of reversals; in other embodiments, a predetermined number of reversals may be in the range of from 4 to 100 reversals. In some embodiments, a plurality of reversals is an even number greater than one. In some embodiments, the plurality of reversals is an even number. In some embodiments, cycles of reversals are carried out; that is, pairs of reversals are carried out. In some embodiments, at least a plurality of cycles of reversals are carried out; or at least two cycles of reversals are carried out; or at least three cycles of reversals are carried out. In some embodiments, the polymers are polynucleotides and the at least one property of the polymers is a nucleotide sequence. In some embodiments, at least two or more different nucleotides of polynucleotides have fluorescent labels that generate distinguishable optical signals from which the identities of the nucleotides may be determined.

In some embodiments, the invention is implemented in an optically-based method of determining characteristics of polymers comprising the following steps: (a) providing a nanopore array comprising a solid phase membrane having a first side, a second side, and a plurality of apertures therethrough, wherein the solid phase membrane separates a first chamber and a second chamber such that each aperture provides fluid communication between the first chamber and the second chamber and wherein each aperture has a detection region; (h) translocating polymers from the first chamber toward the second chamber through the apertures, each polymer having one or more optical labels attached thereto capable of generating an optical signal indicative of a characteristic of the polymer; (c) illuminating the second side of the solid phase membrane so that optical labels in the detection regions generate optical signals; (d) detecting optical signals indicative of characteristics of the polymers from the optical labels in the detection regions to produce polymer data; (e) reversing the translocation of the polymers; (f) repeating steps (c) and (d) to produce redundant polymer data; and (g) determining the characteristics of the polymers from the redundant polymer data. As above, in some embodiments, the polymers are polynucleotides and the at least one property of the polymers is a nucleotide sequence. In some embodiments, a different fluorescent label having a distinct optical signal is attached to different kinds of nucleotide monomers, so that different kinds of nucleotide may be identified by detecting optical signals from the different optical labels. In some embodiments, at least two different kinds of nucleotide are labeled with different fluorescent labels having distinct optical signals.

In some embodiments, polymers may be polynucleotides or proteins. In still other embodiments, polymers may be polynucleotides. In further embodiments, polynucleotides may be single stranded nucleic acids. In some embodiments, a characteristic of polymers analyzed or determined is a monomer sequence, such as a nucleotide sequence, of the polymers. In some embodiments, optical labels on polymers are FRET labels, such as described in U.S. patents and patent and international publications: U.S. Pat. No. 8,771,491; US2013/0203050; or WO2014/190322, which are incorporated herein by reference. In some embodiments, apertures comprise protein nanopores. Briefly, in some embodiments, a FRET label comprises at each detection region at least one FRET donor label and at least one FRET acceptor label, wherein an excitation beam excites the FRET donor labels which, in turn, transfer energy to FRET acceptor labels within a FRET distance of the donor labels which, in turn, emit an optical signal. Typically, an excitation beam comprises a second wavelength and the optical signal comprises a first wavelength distinct from the second wavelength, for example, to permit use of an epi-illumination system. In some embodiments, a detection region may extend from the opaque coating of the first side toward the second side and include an extra-membrane space immediately proximal to the exit of an aperture and/or nanopore. In some embodiments, such extra-membrane space does not extend beyond 50 nm from the exit of a nanopore or aperture; in other embodiments, such extra-membrane space does not extend beyond 10 nm from the exit of a nanopore or aperture.

Briefly, as described more fully in U.S. Pat. No. 8,771,491, in some embodiments, an aperture and/or nanopore may be labeled with one or more FRET donors and polymers may each be labeled with FRET acceptors such that at least selected donors and acceptors form FRET pairs; that is, the emission spectra of a donor overlaps the absorption spectra of at least one acceptor so that if other conditions are met (e.g. donor excitation, donor and acceptor being within a FRET distance, donor and acceptor having proper relative orientation, and the like) a FRET interaction can occur. In a FRET interaction excitation energy of the donor is transferred to an acceptor non-radiatively, after which the acceptor, emits an optical signal that has a lower energy than the excitation energy of the donor. Donor are usually excited by illuminating them with a light beam, such as generated by a laser.

In some embodiments, protein nanopores may be inserted in solid state membranes without, or with only small amounts of, lipid bilayers to form arrays, as described in Huber et al, U.S. patent publication 2013/0203050, which is incorporated herein by reference.

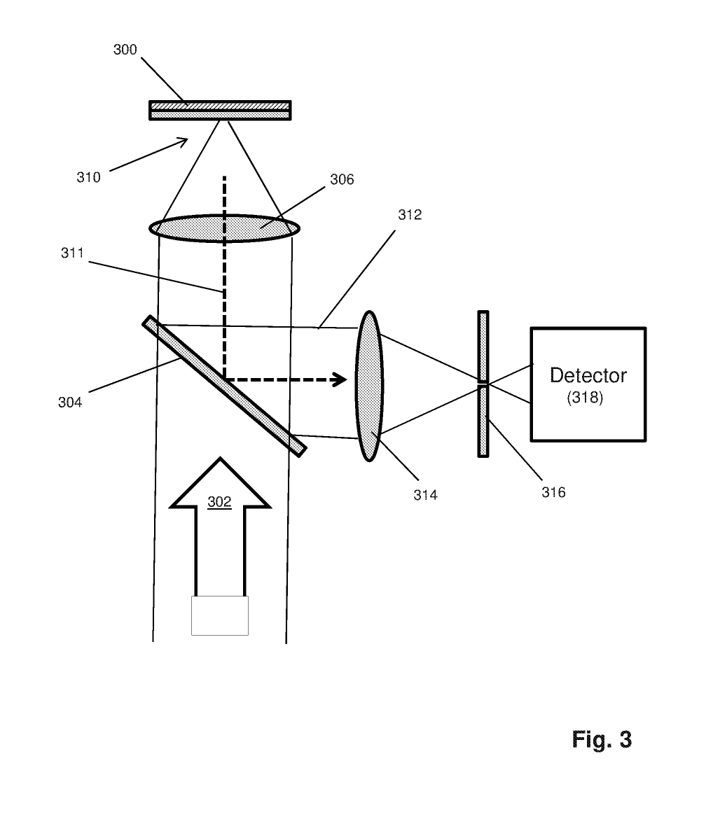

In some embodiments, an epi-illumination system, in which excitation beam delivery and optical signal collection occurs through a single objective, may be used for direct illumination of labels on a polymer analyte or donors on nanopores. The basic components of a confocal epi-illumination system for use with the invention is illustrated in FIG. 3. Excitation beam (302) passes through dichroic (304) and onto objective lens (306) which focuses (310) excitation beam (302) onto layered membrane (300), in which labels are excited directly to emit an optical signal, such as a fluorescent signal, of are excited indirectly via a FRET interaction to emit an optical signal. Such optical signal is collected by objective lens (306) and directed to dichroic (304), which is selected so that it passes light of excitation beam (302) but reflects light of optical signals (311). Reflected optical signals (311) passes through lens (314) which focuses it through pinhole (316) and onto detector (318).

### Controlling Translocation Speed

The role of translocation speed of polynucleotides through nanopores and the need for its control have been appreciated in the field of nanopore technology wherein changes in electric current are use to identify translocating analytes. A wide variety of methods have been used to control translocation speed, which include both methods that can be adjusted in real-time without significant difficulty (e.g. voltage potential across nanopores, temperature, and the like) and methods that can be adjusted during operation only with difficulty (reaction buffer viscosity, presence or absence of charged side chains in the bore of a protein nanopore, ionic composition and concentration of the reaction buffer, velocity-retarding groups attached or hybridized to polynucleotide analytes, molecular motors, and the like), e.g. Bates et al, Biophysical J., 84: 2366-2372 (2003); Carson et al, Nanotechnology, 26(7): 074004 (2015); Yeh et al, Electrophoresis, 33(23): 58-65 (2012); Meller, J. Phys. Cond. Matter, 15: R581-R607 (2003); Luan et al, Nanoscale, 4(4): 1068-1077 (2012); Keyser, J. R. Soc. Interface, 8: 1369-1378 (2011); and the like, which are incorporated herein by reference. In some embodiments, a step or steps are included for active control of translocation speed while a method of the invention is being implemented, e.g. voltage potential, temperature, or the like; in other embodiments, a step or steps are included that determine a translocation speed that is not actively controlled or changed while a method of the invention is being implemented, e.g. reaction buffer viscosity, ionic concentration, and the like. In regard to the latter, in some embodiments, a translocation speed is selected by providing a reaction buffer having a concentration of glycerol, or equivalent reagent, in the range of from 1 to 60 percent.

As mentioned above, translocation speeds depend in part on the voltage difference (or electrical field strength) across a nanopore and conditions in the reaction mixture of the first chamber where nucleic acid polymers are exposed to the nanopore. Nucleic acid polymer capture rates by nanopores depend on concentration of such polymers. In some embodiments, conventional reaction mixture conditions for nanopore sequencing may be employed with the invention, for example, 1M KCl (or equivalent salt, such as NaCl, LiCl, or the like) and a pH buffering system (which, for example, ensures that proteins being used, e.g. protein nanopores, nucleases, or the like, are not denatured). In some embodiments, a pH buffering system may be used to keep the pH substantially constant at a value in the range of 6.8 to 8.8. In some embodiments, a voltage difference across the nanopores may be in the range of from 70 to 300 mV. In other embodiments, a voltage difference across the nanopores may be in the range of from 80 to 200 mV. An appropriate voltage for operation may be selected using conventional measurement techniques. Current (or voltage) across a nanopore may readily be measured using commercially available instruments. A voltage difference may be selected so that translocation speed is within a desired range. In some embodiments, a range of translocation speeds comprises those speeds less than 4000 nucleotides per second. In some embodiments, a range of translocation speeds comprises those speeds less than 1000 nucleotides per second. In other embodiments, a range of translocation speeds is from 10 to 800 nucleotides per second; in other embodiments, a range of translocation speeds is from 10 to 600 nucleotides per second; in other embodiments, a range of translocation speeds is from 200 to 800 nucleotides per second; in other embodiments, a range of translocation speeds is from 200 to 500 nucleotides per second.

In some embodiments, a device for implementing the above methods for single stranded nucleic acids typically includes providing a set of electrodes for establishing an electric field across the nanopores (which may comprise an array). Single stranded nucleic acids are exposed to nanopores by placing them in an electrolyte (i.e. reaction buffer) in a first chamber, which is configured as the “cis” side of the layered membrane by placement of a negative electrode in the chamber. Upon application of an electric field, the negatively single stranded nucleic acids are captured by nanopores and translocated to a second chamber on the other side of the layered membrane, which is configured as the “trans” side of membrane by placement of a positive electrode in the chamber. As mentioned above, the speed of translocation depends in part on the ionic strength of the electrolytes in the first and second chambers and the applied voltage across the nanopores. In optically based detection, a translocation speed may be selected by preliminary calibration measurements, for example, using predetermined standards of labeled single stranded nucleic acids that generate signals at different expected rates per nanopore for different voltages. Thus, for DNA sequencing applications, an initial translocation speed may be selected based on the signal rates from such calibration measurements, as well as the measure based on relative signal intensity distribution discussed above. Consequently, from such measurements a voltage may be selected that permits, or maximizes, reliable nucleotide identifications, for example, over an array of nanopores. In some embodiments, such calibrations may be made using nucleic acids from the sample of templates being analyzed (instead of, or in addition to, predetermined standard sequences). In some embodiments, such calibrations may be carried out in real time during a sequencing run and the applied voltage may be modified in real time based on such measurements, for example, to maximize the acquisition of nucleotide-specific signals.

### Nanopore Arrays

As discussed above, nanopores used with the invention may be solid-state nanopores, protein nanopores, or hybrid nanopores comprising protein nanopores or organic nanotubes such as carbon or graphene nanotubes, configured in a solid-state membrane, or like framework. One function of nanopores is constraining polymer analytes, such as polynucleotides, so that their monomers pass through a detection zone (or signal generation region) in sequence (that is, so that nucleotides pass a detection zone one at a time, or in single file). In accordance with the invention, nanopores are provided in arrays, typically planar arrays. In some embodiments, arrays of nanopores are arranged regularly, for example, in a rectilinear pattern, a hexagonal pattern, or the like. In some embodiments, arrays of nanopores are random arrays, for example, in some embodiments, as described by a Poisson distribution. In some embodiments, nanopores of an array are disposed at known locations. In some embodiments, nanopore arrays include a plurality of nanopores. In some embodiments, such plurality comprises at least 10 nanopores, or in other embodiments, at least 100 nanopores, or in other embodiments, at least 1000 nanopores. In still other embodiments, a nanopore array comprises a plurality of nanopores in the range of from 10 to 10,000. In some embodiments, additional features of nanopores include passing single stranded nucleic acids while not passing double stranded nucleic acids, or equivalently bulky molecules. In some embodiments, additional functions of nanopores include (i) passing single stranded nucleic acids while not passing double stranded nucleic acids, or equivalently bulky molecules and/or (ii) constraining fluorescent labels on nucleotides so that fluorescent signal generation is suppressed or directed so that it is not collected.

In some embodiments, nanopores used in connection with the methods and devices of the invention are provided in the form of arrays, such as an array of clusters of nanopores, which may be disposed regularly or at known locations on a planar surface. In some embodiments, clusters are each in a separate resolution limited area so that optical signals from nanopores of different clusters are distinguishable by the optical detection system employed, but optical signals from nanopores within the same cluster cannot necessarily be assigned to a specific nanopore within such cluster by the optical detection system employed.

Solid state nanopores may be fabricated in a variety of materials including but not limited to, silicon nitride (Si₃N₄), silicon dioxide (SiO₂), and the like. The fabrication and operation of nanopores for analytical applications, such as DNA sequencing, are disclosed in the following exemplary references that are incorporated by reference: Ling, U.S. Pat. No. 7,678,562; Hu et al, U.S. Pat. No. 7,397,232; Golovchenko et al, U.S. Pat. No. 6,464,842; Chu et al, U.S. Pat. No. 5,798,042; Sauer et al, U.S. Pat. No. 7,001,792; Su et al, U.S. Pat. No. 7,744,816; Church et al, U.S. Pat. No. 5,795,782; Bayley et al, U.S. Pat. No. 6,426,231; Akeson et al, U.S. Pat. No. 7,189,503; Bayley et al, U.S. Pat. No. 6,916,665; Akeson et al, U.S. Pat. No. 6,267,872; Meller et al, U.S. patent publication 2009/0029477; Howorka et al, International patent publication WO2009/007743; Brown et al, International patent publication WO2011/067559; Meller et al, International patent publication WO2009/020682; Polonsky et al, International patent publication WO2008/092760; Van der Zaag et al, International patent publication WO2010/007537; Yan et al, Nano Letters, 5(6): 1129-1134 (2005); Iqbal et al, Nature Nanotechnology, 2: 243-248 (2007); Wanunu et al, Nano Letters, 7(6): 1580-1585 (2007); Dekker, Nature Nanotechnology, 2: 209-215 (2007); Storm et al, Nature Materials, 2: 537-540 (2003); Wu et al, Electrophoresis, 29(13): 2754-2759 (2008); Nakane et al, Electrophoresis, 23: 2592-2601 (2002); Zhe et al, J. Micromech. Microeng., 17: 304-313 (2007); Henriquez et al, The Analyst, 129: 478-482 (2004); Jagtiani et al, J. Micromech. Microeng., 16: 1530-1539 (2006); Nakane et al, J. Phys. Condens. Matter, 15 R1365-R1393 (2003); DeBlois et al, Rev. Sci. Instruments, 41(7): 909-916 (1.970); Clarke et al, Nature Nanotechnology, 4(4): 265-270 (2009); Bayley et al, U.S. patent publication 2003/0215881; and the like.

In some embodiments, the invention comprises nanopore arrays with one or more light-blocking layers, that is, one or more opaque layers. Typically nanopore arrays are fabricated in thin sheets of material, such as, silicon, silicon nitride, silicon oxide, aluminum oxide, or the like, which readily transmit light, particularly at the thicknesses used, e.g. less than 50-100 nm. For electrical detection of analytes this is not a problem. However, in optically-based detection of labeled molecules translocating nanopores, light transmitted through an array invariably excites materials outside of intended reaction sites, thus generates optical noise, for example, from nonspecific background fluorescence, fluorescence from labels of molecules that have not yet entered a nanopore, or the like. In one aspect, the invention addresses this problem by providing nanopore arrays with one or more light-blocking layers that reflect and/or absorb light from an excitation beam, thereby reducing background noise for optical signals generated at intended reaction sites associated with nanopores of an array. In some embodiments, this permits optical labels in intended reaction sites to be excited by direct illumination. In some embodiments, an opaque layer may be a metal layer. Such metal layer may comprise Sn, Al, V, Ti, Ni, Mo, Ta, W, Au, Ag or Cu. In some embodiments such metal layer may comprise Al, Au, Ag or Cu. In still other embodiments, such metal layer may comprise aluminum or gold, or may comprise solely aluminum. The thickness of an opaque layer may vary widely and depends on the physical and chemical properties of material composing the layer. In some embodiments, the thickness of an opaque layer may be at least 5 nm, or at least 10 nm, or at least 40 nm. In other embodiments, the thickness of an opaque layer may be in the range of from 5-100 nm; in other embodiments, the thickness of an opaque layer may be in the range of from 10-80 nm. An opaque layer need not block (i.e. reflect or absorb) 100 percent of the light from an excitation beam. In some embodiments, an opaque layer may block at least 10 percent of incident light from an excitation beam; in other embodiments, an opaque layer may block at least 50 percent of incident light from an excitation beam.

Opaque layers or coatings may be fabricated on solid state membranes by a variety of techniques known in the art. Material deposition techniques may be used including chemical vapor deposition, electrodeposition, epitaxy, thermal oxidation, physical vapor deposition, including evaporation and sputtering, casting, and the like. In some embodiments, atomic layer deposition may be used, e.g. U.S. Pat. No. 6,464,842; Wei et al, Small, 6(13): 1406-1414 (2010), which are incorporated by reference.

In some embodiments, a 1-100 nm channel or aperture may be formed through a solid substrate, usually a planar substrate, such as a membrane, through which an analyte, such as single stranded DNA, is induced to translocate. In other embodiments, a 2-50 nm channel or aperture is formed through a substrate; and in still other embodiments, a 2-30 nm, or a 2-20 nm, or a 3-30 nm, or a 3-20 nm, or a 3-10 nm channel or aperture if formed through a substrate. The solid-state approach of generating nanopores offers robustness and durability as well as the ability to tune the size and shape of the nanopore, the ability to fabricate high-density arrays of nanopores on a wafer scale, superior mechanical, chemical and thermal characteristics compared with lipid-based systems, and the possibility of integrating with electronic or optical readout techniques. Biological nanopores on the other hand provide reproducible narrow bores, or lumens, especially in the 1-10 nanometer range, as well as techniques for tailoring the physical and/or chemical properties of the nanopore and for directly or indirectly attaching groups or elements, such as fluorescent labels, which may be FRET donors or acceptors, by conventional protein engineering methods. Protein nanopores typically rely on delicate lipid bilayers for mechanical support, and the fabrication of solid-state nanopores with precise dimensions remains challenging. In some embodiments, solid-state nanopores may be combined with a biological nanopore to form a so-called “hybrid” nanopore that overcomes some of these shortcomings, thereby providing the precision of a biological pore protein with the stability of a solid state nanopore. For optical read out techniques a hybrid nanopore provides a precise location of the nanopore which simplifies the data acquisition greatly.

In some embodiments, clusters may also be formed by disposing protein nanopores in lipid bilayers supported by solid phase membrane containing an array of apertures. For example, such an array may comprise apertures fabricated (e.g. drilled, etched, or the like) in solid phase support. The geometry of such apertures may vary depending on the fabrication techniques employed. In some embodiments, each such aperture is associated with, or encompassed by, a separate resolution limited area; however, in other embodiments, multiple apertures may be within the same resolution limited area. The cross-sectional area of the apertures may vary widely and may or may not be the same as between different clusters, although such areas are usually substantially the same as a result of conventional fabrication approaches. In some embodiments, apertures have a minimal linear dimension (e.g. diameter in the case of circular apertures) in the range of from 10 to 200 nm, or have areas in the range of from about 100 to 3×10⁴ nm². Across the apertures may be disposed a lipid bilayer. The distribution of protein nanopores per aperture may be varied, for example, by controlling the concentration of protein nanopores during inserting step. In such embodiments, clusters of nanopores may comprise a random number of nanopores. In some embodiments, in which protein nanopores insert randomly into apertures, clusters containing one or more apertures on average have a number of protein nanopores that is greater than zero; in other embodiments, such clusters have a number of protein nanopores that is greater than 0.25; in other embodiments, such clusters have a number of protein nanopores that is greater than 0.5; in other embodiments, such clusters have a number of protein nanopores that is greater than 0.75; in other embodiments, such clusters have a number of protein nanopores that is greater than 1.0.

In some embodiments, methods and devices of the invention comprise a solid phase membrane, such as a SiN membrane, having an array of apertures therethrough providing communication between a first chamber and a second chamber (also sometimes referred to as a “cis chamber” and a “trans chamber”) and supporting a lipid bilayer on a surface facing the second, or trans, chamber. In some embodiments, diameters of the aperture in such a solid phase membrane may be in the range of 10 to 200 nm, or in the range of 20 to 100 nm. In some embodiments, such solid phase membranes further include protein nanopores inserted into the lipid bilayer in regions where such bilayer spans the apertures on the surface facing the trans chamber. In some embodiments, such protein nanopores are inserted from the cis side of the solid phase membrane using techniques described herein. In some embodiments, such protein nanopores have a structure identical to, or similar to, α-hemolysin in that it comprises a barrel, or bore, along an axis and at one end has a “cap” structure and at the other end has a “stem” structure (using the terminology from Song et al, Science, 274: 1859-1866 (1996)). In some embodiments using such protein nanopores, insertion into the lipid bilayer results in the protein nanopore being oriented so that its cap structure is exposed to the cis chamber and its stem structure is exposed to the trans chamber.

In some embodiments, the present invention may employ hybrid nanopores in clusters, particularly for optical-based nanopore sequencing of polynucleotides. Such nanopores comprise a solid-state orifice, or aperture, into which a protein biosensor, such as a protein nanopore, is stably inserted. A charged polymer may be attached to a protein nanopore (e.g. alpha hemolysin) by conventional protein engineering techniques after which an applied electric field may be used to guide a protein nanopore, into an aperture in a solid-state membrane. In some embodiments, the aperture in the solid-state substrate is selected to be slightly smaller than the protein, thereby preventing it from translocating through the aperture. Instead, the protein will be embedded into the solid-state orifice.

In some embodiments, a donor fluorophore is attached to the protein nanopore. This complex is then inserted into a solid-state aperture or nanohole (for example, 3-10 nm in diameter) by applying an electric field across the solid state nanohole, or aperture, until the protein nanopore is transported into the solid-state nanohole to form a hybrid nanopore. The formation of the hybrid nanopore can be verified by (a) the inserted protein nanopore causing a drop in current based on a partial blockage of the solid-state nanohole and by (b) the optical detection of the donor fluorophore.

Solid state, or synthetic, nanopores may be prepared in a variety of ways, as exemplified in the references cited above. In some embodiments a helium ion microscope may be used to drill the synthetic nanopores in a variety of materials, e.g. as disclosed by Yang et al, Nanotechnolgy, 22: 285310 (2011), which is incorporated herein by reference. A chip that supports one or more regions of a thin-film material, e.g. silicon nitride, that has been processed to be a free-standing membrane is introduced to the helium ion microscope (HIM) chamber. HIM motor controls are used to bring a free-standing membrane into the path of the ion beam while the microscope is set for low magnification. Beam parameters including focus and stigmation are adjusted at a region adjacent to the free-standing membrane, but on the solid substrate. Once the parameters have been properly fixed, the chip position is moved such that the free-standing membrane region is centered on the ion beam scan region and the beam is blanked. The HIM field of view is set to a dimension (in μm) that is sufficient to contain the entire anticipated nanopore, pattern and sufficient to be useful in future optical readout (i.e. dependent on optical magnification, camera resolution, etc.). The ion beam is then rastered once through the entire field of view at a pixel dwell time that results in a total ion dose sufficient to remove all or most of the membrane autofluorescence. The field of view is then set to the proper value (smaller than that used above) to perform lithographically-defined milling of either a single nanopore or an array of nanopores. The pixel dwell time of the pattern is set to result in nanopores of one or more predetermined diameters, determined through the use of a calibration sample prior to sample processing. This entire process is repeated for each desired region on a single chip and/or for each chip introduced into the HIM chamber.

In some embodiments, a nanopore may have one or more labels attached for use in optically-based nanopore sequencing methods. The label may be a member of a Forster Resonance Energy Transfer (FRET) pair. Such labels may comprise organic fluorophores, chemiluminescent labels, quantum dots, metallic nanoparticles and/or fluorescent proteins. Target nucleic acids may have one distinct label per nucleotide. The labels attached to the nucleotides may be selected from the group consisting of organic fluorophores. The label attachment site in the pore protein can be generated by conventional protein engineering methods, e.g. a mutant protein can be constructed that will allow the specific binding of the label. As an example, a cysteine residue may be inserted at the desired position of the protein which inserts a thiol (SH) group that can be used to attach a label. The cysteine can either replace a natural occurring amino acid or can be incorporated as an addition amino acid. A maleimide-activated label is then covalently attached to the thiol residue of the protein nanopore. In a preferred embodiment the attachment of the label to the protein nanopore or the label on the nucleic acid is reversible. By implementing a cleavable crosslinker, an easily breakable chemical bond (e.g. an S—S bond or a pH labile bond) is introduced and the label may be removed when the corresponding conditions are met.

### Labels for Nanopores and Analytes

In some embodiments, a nanopore may be labeled with one or more quantum dots. In particular, in some embodiments, one or more quantum dots may be attached to a nanopore, or attached to a solid phase support adjacent to (and within a FRET distance of an entrance or exit of a nanopore), and employed as donors in FRET reactions with acceptors on analytes. Such uses of quantum dots are well known and are described widely in the scientific and patent literature, such as, in U.S. Pat. Nos. 6,252,303; 6,855,551; 7,235,361; and the like, which are incorporated herein by reference.

One example of a Quantum dot which may be utilized as a pore label is a CdTe quantum dot which can be synthesized in an aqueous solution. A CdTe quantum dot may be functionalized with a nucleophilic group such as primary amines, thiols or functional groups such as carboxylic acids. A CdTe quantum dot may include a mercaptopropionic acid capping ligand, which has a carboxylic acid functional group that may be utilized to covalently link a quantum dot to a primary amine on the exterior of a protein pore. The cross-linking reaction may be accomplished using standard cross-linking reagents (homo-bifunctional as well as hetero-bifunctional) which are known to those having ordinary skill in the art of bioconjugation. Care may be taken to ensure that the modifications do not impair or substantially impair the translocation of a nucleic acid through the nanopore. This may be achieved by varying the length of the employed crosslinker molecule used to attach the donor label to the nanopore.

For example, the primary amine of the lysine residue 131 of the natural alpha hemolysin protein (Song, L. et al., Science 274, (1996): 1859-1866) may be used to covalently bind carboxy modified CdTe Quantum dots via 1-Ethyl-3-[3-dimethylaminopropyl]carbodiimide hydrochloride/N-hydroxysulfosuccinimide (EDC/NHS) coupling chemistry. Alternatively, amino acid 129 (threonine) may be exchanged into cysteine. Since there is no other cysteine residue in the natural alpha hemolysin protein the thiol side group of the newly inserted cysteine may be used to covalently attach other chemical moieties.

A biological polymer, e.g., a nucleic acid molecule or polymer, may be labeled with one or more acceptor labels. For a nucleic acid molecule, each of the four nucleotides or building blocks of a nucleic acid molecule may be labeled with an acceptor label thereby creating a labeled (e.g., fluorescent) counterpart to each naturally occurring nucleotide. The acceptor label may be in the form of an energy accepting molecule which can be attached to one or more nucleotides on a portion or on the entire strand of a converted nucleic acid.

A variety of methods may be utilized to label the monomers or nucleotides of a nucleic acid molecule or polymer. A labeled nucleotide may be incorporated into a nucleic acid during synthesis of a new nucleic acid using the original sample as a template (“labeling by synthesis”). For example, the labeling of nucleic acid may be achieved via PCR, whole genome amplification, rolling circle amplification, primer extension or the like or via various combinations and extensions of the above methods known to persons having ordinary skill in the art.

A label may comprise a reactive group such as a nucleophile (amines, thiols etc.). Such nucleophiles, which are not present in natural nucleic acids, can then be used to attach fluorescent labels via amine or thiol reactive chemistry such as NHS esters, maleimides, epoxy rings, isocyanates etc. Such nucleophile reactive fluorescent dyes (i.e. NHS-dyes) are readily commercially available from different sources. An advantage of labeling a nucleic acid with small nucleophiles lies in the high efficiency of incorporation of such labeled nucleotides when a “labeling by synthesis” approach is used. Bulky fluorescently labeled nucleic acid building blocks may be poorly incorporated by polymerases due to steric hindrance of the labels during the polymerization process into newly synthesized DNA.

Whenever two or more mutually quenching dyes are used, such dyes may be attached to DNA using orthogonal attachment chemistries. For example, NHS esters can be used to react very specifically with primary amines or maleimides will react with thiol groups. Either primary amines (NH₂) or thiol (SH) modified nucleotides are commercially available. These relatively small modifications are readily incorporated in a polymerase mediated DNA synthesis and can be used for subsequent labeling reactions using either NHS or maleimide modified dyes. Guidance for selecting and using such orthogonal linker chemistries may be found in Hermanson (cited above).

Additional orthogonal attachment chemistries for typical attachment positions include Huisgen-type cycloaddition for a copper-catalyzed reaction and an uncatalyzed reaction; alkene plus nitrile oxide cycloaddition, e.g. as disclosed in Gutsmiedl et al, Org. Lett., 11: 2405-2408 (2009); Diels-Alder cycloaddition, e.g. disclosed in Seelig et al, Tetrahedron Lett., 38: 7729-7732 (1997); carbonyl ligation, e.g. as disclosed in Casi et al, J. Am. Chem. Soc., 134: 5887-5892 (2012); Shao et al J. Am. Chem. Soc., 117: 3893-3899 (1995); Rideout, Science, 233: 561-563 (1986); Michael addition, e.g. disclosed in Brinkley, Bioconjugate Chemistry, 3: 2-13 (1992); native chemical ligation, e.g. disclosed in Schuler et al, Bioconjugate Chemistry, 13: 1039-1043 (2002); Dawson et al, Science, 266: 776-779 (1994); or amide formation via an active ester, e.g. disclosed in Hermanson (cited above).

A combination of 1, 2, 3 or 4 nucleotides in a nucleic acid strand may be exchanged with their labeled counterpart. The various combinations of labeled nucleotides can be sequenced in parallel, e.g., labeling a source nucleic acid or DNA with combinations of 2 labeled nucleotides in addition to the four single labeled samples, which will result in a total of 10 differently labeled sample nucleic acid molecules or DNAs (G, A, T, C, GA, GT, GC, AT, AC, TC). The resulting sequence pattern may allow for a more accurate sequence alignment due to overlapping nucleotide positions in the redundant sequence read-out. In some embodiments, a polymer, such as a polynucleotide or polypeptide, may be labeled with a single fluorescent label attached to a single kind of monomer, for example, every T (or substantially every T) of a polynucleotide is labeled with a fluorescent label, e.g. a cyanine dye. In such embodiments, a collection, or sequence, of fluorescent signals from the polymer may form a signature or fingerprint for the particular polymer. In some such embodiments, such fingerprints may or may not provide enough information for a sequence of monomers to be determined.

In some embodiments, a feature of the invention is the labeling of substantially all monomers of a polymer analyte with fluorescent dyes or labels that are members of a mutually quenching set. The use of the term “substantially all” in reference to labeling polymer analytes is to acknowledge that chemical and enzymatic labeling techniques are typically less than 100 percent efficient. In some embodiments, “substantially all” means at least 80 percent of all monomer have fluorescent labels attached. In other embodiments, “substantially all” means at least 90 percent of all monomer have fluorescent labels attached. In other embodiments, “substantially all” means at least 95 percent of all monomer have fluorescent labels attached.

A method for sequencing a polymer, such as a nucleic acid molecule includes providing a nanopore or pore protein (or a synthetic pore) inserted in a membrane or membrane like structure or other substrate. The base or other portion of the pore may be modified with one or more pore labels. The base may refer to the Trans side of the pore. Optionally, the Cis and/or Trans side of the pore may be modified with one or more pore labels. Nucleic acid polymers to be analyzed or sequenced may be used as a template for producing a labeled version of the nucleic acid polymer, in which one of the four nucleotides or up to all four nucleotides in the resulting polymer is/are replaced with the nucleotide's labeled analogue(s). An electric field is applied to the nanopore which forces the labeled nucleic acid polymer through the nanopore, while an external monochromatic or other light source may be used to illuminate the nanopore, thereby exciting the pore label. As, after or before labeled nucleotides of the nucleic acid pass through, exit or enter the nanopore, energy is transferred from the pore label to a nucleotide label, which results in emission of lower energy radiation. The nucleotide label radiation is then detected by a confocal microscope setup or other optical detection system or light microscopy system capable of single molecule detection known to people having ordinary skill in the art. Examples of such detection systems include but are not limited to confocal microscopy, epi-illumination fluorescence microscopy, total internal reflection fluorescent (TIRF) microscopy, and the like. In some embodiments, epi-illumination fluorescence microscopy is employed.

Energy may be transferred from a pore or nanopore donor label (e.g., a Quantum Dot) to an acceptor label on a polymer (e.g., a nucleic acid) when an acceptor label of an acceptor labeled monomer (e.g., nucleotide) of the polymer interacts with the donor label as, after or before the labeled monomer exits, enters or passes through a nanopore. For example, the donor label may be positioned on or attached to the nanopore on the cis or trans side or surface of the nanopore such that the interaction or energy transfer between the donor label and acceptor label does not take place until the labeled monomer exits the nanopore and comes into the vicinity or proximity of the donor label outside of the nanopore channel or opening. As a result, interaction between the labels, energy transfer from the donor label to the acceptor label, emission of energy from the acceptor label and/or measurement or detection of an emission of energy from the acceptor label may take place outside of the passage, channel or opening running through the nanopore, e.g., within a cis or trans chamber on the cis or trans sides of a nanopore. The measurement or detection of the energy emitted from the acceptor label of a monomer may be utilized to identify the monomer.

The nanopore label may be positioned outside of the passage, channel or opening of the nanopore such that the label may be visible or exposed to facilitate excitation or illumination of the label. The interaction and energy transfer between a donor label and accepter label and the emission of energy from the acceptor label as a result of the energy transfer may take place outside of the passage, channel or opening of the nanopore. This may facilitate ease and accuracy of the detection or measurement of energy or light emission from the acceptor label, e.g., via an optical detection or measurement device.

A donor label may be attached in various manners and/or at various sites on a nanopore. For example, a donor label may be directly or indirectly attached or connected to a portion or unit of the nanopore. Alternatively, a donor label may be positioned adjacent to a nanopore.

Each acceptor labeled monomer (e.g., nucleotide) of a polymer (e.g., nucleic acid) can interact sequentially with a donor label positioned on or next to or attached directly or indirectly to the exit of a nanopore or channel through which the polymer is translocated. The interaction between the donor and acceptor labels may take place outside of the nanopore channel or opening, e.g., after the acceptor labeled monomer exits the nanopore or before the monomer enters the nanopore. The interaction may take place within or partially within the nanopore channel or opening, e.g., while the acceptor labeled monomer passes through, enters or exits the nanopore.

When one of the four nucleotides of a nucleic acid is labeled, the time dependent signal arising from the single nucleotide label emission is converted into a sequence corresponding to the positions of the labeled nucleotide in the nucleic acid sequence. The process is then repeated for each of the four nucleotides in separate samples and the four partial sequences are then aligned to assemble an entire nucleic acid sequence.

When multi-color labeled nucleic acid (DNA) sequences are analyzed, the energy transfer from one or more donor labels to each of the four distinct acceptor labels that may exist on a nucleic acid molecule may result in light emission at four distinct wavelengths or colors (each associated with one of the four nucleotides) which allows for a direct sequence read-out.

A donor label (also sometimes referred to herein as a “pore label”) may be placed as close as possible to the aperture (for example, at the exit) of a nanopore without causing an occlusion that impairs translocation of a nucleic acid through the nanopore. A pore label may have a variety of suitable properties and/or characteristics. For example, a pore label may have energy absorption properties meeting particular requirements. A pore label may have a large radiation energy absorption cross-section, ranging, for example, from about 0 to 1000 nm or from about 200 to 500 nm. A pore label may absorb radiation within a specific energy range that is higher than the energy absorption of the nucleic acid label, such as an acceptor label. The absorption energy of the pore label may be tuned with respect to the absorption energy of a nucleic acid label in order to control the distance at which energy transfer may occur between the two labels. A pore label may be stable and functional for at least 106 to 109 excitation and energy transfer cycles.

In some embodiments, a device for analyzing polymers each having optical labels attached to a sequence of monomers may comprise the following elements: (a) a nanopore array in a solid phase membrane separating a first chamber and a second chamber, wherein nanopores of the nanopore array each provide fluid communication between the first chamber and the second chamber and are arranged in clusters such that each different cluster of nanopores is disposed within a different resolution limited area and such that each cluster comprises a number of nanopores that is either greater than one or is a random variable with an average value greater than zero; (b) a polymer translocating system for moving polymers in the first chamber to the second chamber through the nanopores of the nanopore array; and (c) a detection system for collecting optical signals generated by optical labels attached to polymers whenever an optical label exits a nanopore within a resolution limited area.

### Definitions

“FRET” or “Förster, or fluorescence, resonant energy transfer” means a non-radiative dipole-dipole energy transfer mechanism from an excited donor fluorophore to an acceptor fluorophore in a ground state. The rate of energy transfer in a FRET interaction depends on the extent of spectral overlap of the emission spectrum of the donor with the absorption spectrum of the acceptor, the quantum yield of the donor, the relative orientation of the donor and acceptor transition dipoles, and the distance between the donor and acceptor molecules, Lakowitz, Principles of Fluorescence Spectroscopy, Third Edition (Springer, 2006). FRET interactions of particular interest are those which result a portion of the energy being transferred to an acceptor, in turn, being emitted by the acceptor as a photon, with a frequency lower than that of the light exciting its donor (i.e. a “FRET signal”). “FRET distance” means a distance between a FRET donor and a FRET acceptor over which a FRET interaction can take place and a detectable FRET signal produced by the FRET acceptor.

“Nanopore” means any opening positioned in a substrate that allows the passage of analytes through the substrate in a predetermined or discernable order, or in the case of polymer analytes, passage of their monomeric units through the substrate in a pretermined or discernible order. In the latter case, a predetermined or discernible order may be the primary sequence of monomeric units in the polymer. Examples of nanopores include proteinaceous or protein based nanopores, synthetic or solid state nanopores, and hybrid nanopores comprising a solid state nanopore having a protein nanopore embedded therein. A nanopore may have an inner diameter of 1-10 nm or 1-5 nm or 1-3 nm. Examples of protein nanopores include but are not limited to, alpha-hemolysin, voltage-dependent mitochondrial porin (VDAC), OmpF, OmpC, MspA and LamB (maltoporin), e.g. disclosed in Rhee, M. et al., Trends in Biotechnology, 25(4) (2007): 174-181; Bayley et al (cited above); Gundlach et al, U.S. patent publication 2012/0055792; and the like, which are incorporated herein by reference. Any protein pore that allows the translocation of single nucleic acid molecules may be employed. A nanopore protein may be labeled at a specific site on the exterior of the pore, or at a specific site on the exterior of one or more monomer units making up the pore forming protein. Pore proteins are chosen from a group of proteins such as, but not limited to, alpha-hemolysin, MspA, voltage-dependent mitochondrial porin (VDAC), Anthrax porin, OmpF, OmpC and LamB (maltoporin). Integration of the pore protein into the solid state hole is accomplished by attaching a charged polymer to the pore protein. After applying an electric field the charged complex is electrophoretically pulled into the solid state hole. A synthetic nanopore, or solid-state nanopore, may be created in various forms of solid substrates, examples of which include but are not limited to silicones (e.g. Si3N4, SiO2), metals, metal oxides (e.g. Al2O3) plastics, glass, semiconductor material, and combinations thereof. A synthetic nanopore may be more stable than a biological protein pore positioned in a lipid bilayer membrane. A synthetic nanopore may also be created by using a carbon nanotube embedded in a suitable substrate such as but not limited to polymerized epoxy. Carbon nanotubes can have uniform and well-defined chemical and structural properties. Various sized carbon nanotubes can be obtained, ranging from one to hundreds of nanometers. The surface charge of a carbon nanotube is known to be about zero, and as a result, electrophoretic transport of a nucleic acid through the nanopore becomes simple and predictable (Ito, T. et al., Chem. Commun. 12 (2003): 1482-83). The substrate surface of a synthetic nanopore may be chemically modified to allow for covalent attachment of the protein pore or to render the surface properties suitable for optical nanopore sequencing. Such surface modifications can be covalent or non-covalent. Most covalent modification include an organosilane deposition for which the most common protocols are described: 1) Deposition from aqueous alcohol. This is the most facile method for preparing silylated surfaces. A 95% ethanol-5% water solution is adjusted to pH 4.5-5.5 with acetic acid. Silane is added with stiffing to yield a 2% final concentration. After hydrolysis and silanol group formation the substrate is added for 2-5 min. After rinsed free of excess materials by dipping briefly in ethanol. Cure of the silane layer is for 5-10 min at 110 degrees Celsius, 2) Vapor Phase Deposition. Silanes can be applied to substrates under dry aprotic conditions by chemical vapor deposition methods. These methods favor monolayer deposition. In closed chamber designs, substrates are heated to sufficient temperature to achieve 5 mm vapor pressure. Alternatively, vacuum can be applied until silane evaporation is observed. 3) Spin-on deposition. Spin-on applications can be made under hydrolytic conditions which favor maximum functionalization and polylayer deposition or dry conditions which favor monolayer deposition. In some embodiments, single nanopores are employed with methods of the invention. In other embodiments, a plurality of nanopores are employed. In some of the latter embodiments, a plurality of nanopores is employed as an array of nanopores, usually disposed in a planar substrate, such as a solid phase membrane. Nanopores of a nanopore array may be spaced regularly, for example, in a rectilinear pattern, or may be spaced randomly. In a preferred embodiment, nanopores are spaced regularly in a rectilinear pattern in a planar solid phase substrate.

“Peptide,” “peptide fragment,” “polypeptide,” “oligopeptide,” or “fragment” in reference to a peptide are used synonymously herein and refer to a compound made up of a single unbranched chain of amino acid residues linked by peptide bonds. Amino acids in a peptide or polypeptide may be derivatized with various moieties, including but not limited to, polyethylene glycol, dyes, biotin, haptens, or like moieties. The number of amino acid residues in a protein or polypeptide or peptide may vary widely; however, in some embodiments, protein or polypeptides or peptides referred to herein may have 2 from to 70 amino acid residues; and in other embodiments, they may have from 2 to 50 amino acid residues. In other embodiments, proteins or polypeptides or peptides referred to herein may have from a few tens of amino acid residues, e.g. 20, to up to a thousand or more amino acid residues, e.g. 1200. In still other embodiments, proteins, polypeptides, peptides, or fragments thereof, may have from 10 to 1000 amino acid residues; or they may have from 20 to 500 amino acid residues; or they may have from 20 to 200 amino acid residues.

“Polymer” means a plurality of monomers connected into a linear chain. Usually, polymers comprise more than one type of monomer, for example, as a polynucleotide comprising A's, C's, G's and T's, or a polypeptide comprising more than one kind of amino acid. Monomers may include without limitation nucleosides and derivatives or analogs thereof and amino acids and derivatives and analogs thereof. In some embodiments, polymers are polynucleotides, whereby nucleoside monomers are connected by phosphodiester linkages, or analogs thereof.

“Polynucleotide” or “oligonucleotide” are used interchangeably and each mean a linear polymer of nucleotide monomers. Monomers making lap polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moieties, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or internucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references. Likewise, the oligonucleotide and polynucleotide may refer to either a single stranded form or a double stranded form (i.e. duplexes of an oligonucleotide or polynucleotide and its respective complement). It will be clear to one of ordinary skill which form or whether both forms are intended from the context of the terms usage.

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references that are incorporated by reference: Dieffenbach, editor, PCR Primer: A Laboratory Manual, 2nd Edition (Cold Spring Harbor Press, New York, 2003).

“Sequence determination”, “sequencing” or “determining a nucleotide sequence” or like terms in reference to polynucleotides includes determination of partial as well as full sequence information of the polynucleotide. That is, the terms include sequences of subsets of the full set of four natural nucleotides, A, C, G and T, such as, for example, a sequence of just A's and C's of a target polynucleotide. That is, the terms include the determination of the identities, ordering, and locations of one, two, three or all of the four types of nucleotides within a target polynucleotide. In some embodiments, the terms include the determination of the identities, ordering, and locations of two, three or all of the four types of nucleotides within a target polynucleotide. In some embodiments sequence determination may be accomplished by identifying the ordering and locations of a single type of nucleotide, e.g. cytosines, within the target polynucleotide “catcgc . . . ” so that its sequence is represented as a binary code, e.g. “100101 . . . ” representing “c-(not c)(not c)c-(not c)-c . . . ” and the like. In some embodiments, the terms may also include subsequences of a target polynucleotide that serve as a fingerprint for the target polynucleotide; that is, subsequences that uniquely identify a target polynucleotide, or a class of target polynucleotides, within a set of polynucleotides, e.g. all different RNA sequences expressed by a cell.

This disclosure is not intended to be limited to the scope of the particular forms set forth, but is intended to cover alternatives, modifications, and equivalents of the variations described herein. Further, the scope of the disclosure fully encompasses other variations that may become obvious to those skilled in the art in view of this disclosure. The scope of the present invention is limited only by the appended claims.

What is claimed is:

1. A method of analyzing characteristics of polymers by a nanopore array comprising:
(a) providing a nanopore array wherein each nanopore is capable of providing fluid communication between a first chamber and a second chamber and providing signals related to at least one property of a polymer translocating therethrough and wherein a fraction of nanopores in the nanopore array contains polymers;
(b) translocating polymers through nanopores of the nanopore array from the first chamber to the second chamber;
(c) detecting forward signals from the translocating polymers;
(d) reversing the translocation of polymers;
(e) detecting reverse signals from polymers whose translocation through a nanopore was reversed;
(f) determining at least one property of each such polymer from the forward and reverse signals.

2. The method of claim 1 wherein said steps (b) through (e) are repeated.

3. The method of claim 2 wherein said steps (b) through (e) are repeated until either said fraction of nanopores having polymers drops below a predetermined level or a predetermined number of reversals is reached, whichever occurs first.

4. The method of claim 3 wherein said fraction of nanopores having polymers is determined as a function of total current through said nanopore array and/or a function of total optical signal collected from all nanopores in said nanopore array whenever said forward and reverse signals are optical signals.

5. The method of claim 2 wherein durations of said steps (b) and (c) are substantially equal to durations of said steps (d) and (e).

6. The method of claim 1 wherein said polymers have free ends so that each is capable of moving from said first chamber to said second chamber through a nanopore of said nanopore array.

7. The method of claim 2 wherein said polymers are polynucleotides and said at least one property is a nucleotide sequence.

8. The method of claim 7 wherein different kinds of nucleotides of said polynucleotide have different fluorescent labels attached which generate distinguishable optical signals, so that different kinds of nucleotide may be identified by an optical signal from its attached fluorescent label.

9. The method of claim 7 wherein said polynucleotides translocating said nanopores form random coils in said first chamber and said second chamber.

10. The method of claim 9 wherein each of said polynucleotides has a length of at least 1000 nucleotides.

11. The method of claim 2 wherein said polymers are polypeptides and said at least one property is a peptide sequence.

12. The method of claim 11 wherein at least two different kinds of amino acid residues of said polypeptide have different fluorescent labels attached which generate distinguishable optical signals, so that the different kinds of labeled amino acid residues may be identified by an optical signal from its attached fluorescent label.

13. A method of determining characteristics of polymers, the method comprising:
(a) providing a nanopore array comprising a solid phase membrane having a first side, a second side, and a plurality of apertures therethrough each comprising at least one nanopore, wherein the solid phase membrane separates a first chamber and a second chamber such that each nanopore provides fluid communication between the first chamber and the second chamber and wherein each nanopore has a detection region on the second side of the solid phase membrane;
(b) translocating polymers from the first chamber toward the second chamber through the nanopores, each polymer having one or more optical labels attached thereto capable of generating an optical signal indicative of a characteristic of the polymer;
(c) illuminating the second side of the solid phase membrane so that optical labels in the detection regions generate optical signals;
(d) detecting optical signals indicative of characteristics of the polymers from the optical labels in the detection regions to produce polymer data;
(e) reversing the translocation of the polymers;
(f) repeating steps (c) and (d) to produce redundant polymer data; and
(g) determining the characteristics of the polymers from the polymer data and the redundant polymer data.

14. The method of claim 13 wherein said steps (e) and (I) are repeated.

15. The method of claim 14 wherein said polymers are polynucleotides and said at least one property is a nucleotide sequence.

16. The method of claim 15 wherein different kinds of nucleotides of said polynucleotide have different fluorescent labels attached which generate distinguishable optical signals, so that different kinds of nucleotide may be identified by an optical signal from its attached fluorescent label.

17. The method of claim 16 wherein said polynucleotides translocating said nanopores form random coils in said first chamber and said second chamber.

18. The method of claim 17 wherein each of said polynucleotides has a length of at least 1000 nucleotides.

19. A method of determining nucleotide sequences of polynucleotides by a nanopore array comprising:
(a) providing a nanopore array wherein each nanopore is capable of providing fluid communication between a first chamber and a second chamber and providing polynucleotides whose different kinds of nucleotides have different fluorescent labels attached which generate distinguishable optical signals, so that different kinds of nucleotide may be identified by an optical signal from its attached fluorescent label and wherein a fraction of nanopores in the nanopore array are occupied by polynucleotides;
(b) translocating polynucleotides through nanopores of the nanopore array in a direction from the first chamber to the second chamber;
(c) detecting forward optical signals from the translocating polynucleotides;
(d) reversing the direction of translocation of the polynucleotides;
(e) detecting reverse optical signals from the polynucleotides whose translocation through a nanopore was reversed; and
(f) determining a nucleotide sequence of each polynucleotide from the forward and reverse optical signals.

20. The method of claim 19 further including a step of repeating said steps (b) through (e).

21. The method of claim 20 wherein said polynucleotides translocating said nanopores form random coils in said first chamber and said second chamber.

22. The method of claim 21 wherein each of said polynucleotides has a length of at least 1000 nucleotides.

23. The method of claim 20 wherein said steps (b) through (e) are repeated until either said fraction of nanopores having said polynucleotides falls below a predetermined level or a predetermined number of repetitions is reached, whichever occurs first.

24. The method of claim 23 wherein said fraction of nanopores having polynucleotides is determined as a function of total current through said nanopore array and/or a function of total optical signal collected from all nanopores in said nanopore array.
